(19) United States
(12) Patent Application Publication
(10) Pub. No.: US 2024/0366564 A1
(43) Pub. Date: Nov. 7, 2024

(54) Use of Chlorophyll Derivatives for the Treatment of SARS-CoV-2 Infection

(71) Applicant: AffinityImmuno Inc., Charlottetown (CA)

(72) Inventors: Jonathan Zuccolo, Brackley (CA); Amir J Zuccolo, Brackley (CA); Jimmy D Dikeakos, London (CA); Corby Fink, Ilderton (CA); Gregory A Dekaban, London (CA)

(21) Appl. No.: 18/558,411

(22) PCT Filed: May 4, 2022
(86) PCT No.: PCT/CA2022/050697

§ 371 Date: Nov. 1, 2023

Related U.S. Application Data
(60) Provisional application No. 63/201,568, filed on May 5, 2021.

(51) Int. Cl. A61K 31/409; A61K 36/00; A61P 31/14

(52) U.S. Cl. CPC A61K 31/409; A61K 36/00; A61P 31/14

(57) ABSTRACT

The present application relates to the use of pheophorbide A, pheophorbide A analog, or pharmaceutically acceptable salt thereof for blocking the entry of SARS-CoV-2 in an ACE2-expressing cell, for treating an infection by SARS-CoV-2 and/or related disease (COVID-19), for reducing the risk of developing COVID-19 or the severity of COVID-19, in a subject. The pheophorbide A, pheophorbide A analog, or pharmaceutically acceptable salt thereof may be present in an extract, such as a plant or algae extract, or in purified form.

### CROSS REFERENCE TO RELATED APPLICATIONS

The present application claims the benefit of U.S. provisional patent application No. 63/201,568 filed on May 5, 2021, which is incorporated herein by reference.

### TECHNICAL FIELD

The present disclosure generally relates to viral infection, and more particularly to the prevention and/or treatment of coronavirus infection, such as severe acute respiratory syndrome coronavirus 2 (SARS-CoV-2) infection, and related diseases.

### BACKGROUND ART

Coronaviruses are large, roughly spherical RNA viruses with bulbous surface projections that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause Severe Acute Respiratory Syndrome (SARS), Middle East Respiratory Syndrome (MERS), and Coronavirus disease 2019 (COVID-19). Coronaviruses have four structural proteins, namely the Spike (S), Envelope (E), and Membrane (M) proteins, within the viral envelope, as well as containing the Nucleocapsid (N) protein, that encapsulates the viral RNA genome.

Severe acute respiratory syndrome coronavirus 2 (SARS-CoV-2) is the type of coronavirus that causes COVID-19, the respiratory illness responsible for the COVID-19 pandemic. The clinical manifestations of this disease are pneumonia, lung injury, inflammation, and severe acute respiratory syndrome (SARS). The spike protein of SARS-CoV-2 is the glycoprotein responsible for allowing the virus to attach to and fuse with the membrane of a host cell; specifically, its S1 subunit drives attachment to the receptor on the cell, and its S2 subunit drives the fusion between the viral membrane and the cell's plasma membrane. The main receptor involved in SARS-CoV-2 entry into human cells is the angiotensin converting enzyme 2 (ACE2). After attachment of a SARS-CoV-2 virion to a target cell, the cell's protease transmembrane protease, serine 2 (TMPRSS2) cuts open the spike protein of the virus, exposing a fusion peptide in the S2 subunit.

Multiple variants (strains) of SARS-CoV-2 are circulating globally and within the United States. New variants have rapidly become dominant within these countries and have aroused concerns: B.1.1.7 (also known as VOC-202012/01 or Alpha), 501Y.V2 (B.1.351 or Beta), P.1 (B.1.1.28.1 or Gamma), Delta (B.1.617.2) and B.1.1.529 (Omicron, which includes BA.1, BA.2 and BA.3 sublineages).

The B.1.1.7 variant (23 mutations with 17 amino acid changes) was first described in the United Kingdom in December 2020; the 501Y.V2 variant (23 mutations with 17 amino acid changes) was initially reported in South Africa in December 2020; and the P.1 variant (approximately 35 mutations with 17 amino acid changes) was reported in Brazil in January 2021. By February 2021, the B.1.1.7 variant had been reported in 93 countries, the 501Y.V2 variant in 45, and the P.1 variant in 21. All three variants have the N501Y mutation, which changes the amino acid asparagine (N) to tyrosine (Y) at position 501 in the receptor-binding domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity). Recently, two more SARS-CoV-2 variants, B.1.427 and B.1.429, which were first detected in California, have been shown to be approximately 20% more transmissible than pre-existing variants and have been classified by the CDC as variants of concern. The B.1.617.2 Delta variant comprises the following substitutions in the Spike protein that are known to affect transmissibility of the virus: D614G, T478K, P681R and L452R. The B.1.1.529 (Omicron) variant was reported to the WHO in November 2021 and comprises 32 mutations in the Spike protein. Studies on these variants have provided compelling evidence that they have the potential to escape naturally-induced immunity as well as the immunity induced by currently approved vaccines.

Vaccine approaches from multiple companies are showing signs of reducing the severity of the infection although, at the current time, global infection rates are at their highest since the beginning of this pandemic, indicating that the current rate of vaccination is not yet approaching global herd immunity. Furthermore, the emerging SARS-CoV-2 variants exhibit several distinct genomic and structural mutations reducing the efficacy of the vaccines. Thus, current evidence suggests that SARS-CoV-2 will become endemic in the population.

Thus, there is a need for the development of alternative therapies, such as novel inhibitors that can interfere with viral entry or viral propagation, for the management of SARS-CoV-2 infection and COVID-19.

The present description refers to a number of documents, the content of which is herein incorporated by reference in their entirety.

### SUMMARY

The present disclosure provides the following items 1 to 33:
1. A method for blocking the entry and/or replication of severe acute respiratory syndrome coronavirus-2 (SARS-CoV-2) in an ACE2-expressing cell, the method comprising contacting the cell with an effective amount of pheophorbide A, a pheophorbide A analog, or a pharmaceutically acceptable salt thereof.
2. A method for treating an infection by SARS-CoV-2 in a subject comprising administering to said subject an effective amount of pheophorbide A, a pheophorbide A analog, or a pharmaceutically acceptable salt thereof.
3. A method for preventing or treating COVID-19 in a subject comprising administering to said subject an effective amount of pheophorbide A, a pheophorbide A analog, or a pharmaceutically acceptable salt thereof.
4. A method for reducing the risk of developing COVID-19 or the severity of COVID-19 in a subject, the method comprising administering to said subject an effective amount of pheophorbide A, a pheophorbide A analog, or a pharmaceutically acceptable salt thereof.
5. The method of any one of items 1 to 4, wherein an effective amount of pheophorbide A is administered.
6. The method of any one of items 1 to 5, wherein the pheophorbide A, pheophorbide A analog, or pharmaceutically acceptable salt thereof is present in an extract.
7. The method of item 6, wherein the extract is a plant or algae extract.
8. The method of any one of items 1 to 5, wherein the pheophorbide A, pheophorbide A analog, or pharmaceutically acceptable salt thereof is in purified form.
9. The method of any one of items 1 to 8, wherein the pheophorbide A, pheophorbide A analog, or pharmaceutically acceptable salt thereof is formulated into a pharmaceutical composition.
10. The method of any one of items 1 to 9, wherein the method comprises administering a pharmaceutical composition comprising pheophorbide A.
11. The method of any one of items 1 to 10, wherein the pheophorbide A, pheophorbide A analog, or pharmaceutically acceptable salt thereof is administered intrapulmonary.
12. The method of any one of items 1 to 11, wherein the subject is a human.
13. The method of any one of items 1 to 11, wherein the subject is a non-human animal.
14. The method of item 13, wherein the non-human animal is a farm animal.
15. The method of item 13, wherein the non-human animal is a pet.
16. Use of pheophorbide A, a pheophorbide A analog, or a pharmaceutically acceptable salt thereof for blocking the entry and/or replication of SARS-CoV-2 in an ACE2-expressing cell.
17. Use of pheophorbide A, a pheophorbide A analog, or a pharmaceutically acceptable salt thereof for the manufacture of a medicament blocking the entry and/or replication of SARS-CoV-2 in an ACE2-expressing cell.
18. Use of pheophorbide A, a pheophorbide A analog, or a pharmaceutically acceptable salt thereof for treating an infection by SARS-CoV-2 in a subject.
19. Use of pheophorbide A, a pheophorbide A analog, or a pharmaceutically acceptable salt thereof for the manufacture of a medicament for treating an infection by SARS-CoV-2 in a subject.
20. Use of pheophorbide A, a pheophorbide A analog, or a pharmaceutically acceptable salt thereof for treating COVID-19 in a subject.
21. Use of pheophorbide A, a pheophorbide A analog, or a pharmaceutically acceptable salt thereof for the manufacture of a medicament for treating COVID-19 in a subject.
22. Use of pheophorbide A, a pheophorbide A analog, or a pharmaceutically acceptable salt thereof for reducing the risk of developing COVID-19, or the severity of COVID-19 in a subject.
23. Use of pheophorbide A, a pheophorbide A analog, or a pharmaceutically acceptable salt thereof for the manufacture of a medicament for reducing the risk of developing COVID-19, or the severity of COVID-19 in a subject.
24. The use of any one of items 16 to 23, wherein pheophorbide A is used.
25. The use of any one of items 16 to 24, wherein the pheophorbide A, pheophorbide A analog, or pharmaceutically acceptable salt thereof is present in an extract.
26. The use of item 25, wherein the extract is a plant or algae extract.
27. The use of any one of items 16 to 26, wherein the pheophorbide A, pheophorbide A analog, or pharmaceutically acceptable salt thereof is in purified form.
28. The use of any one of items 16 to 27, wherein the pheophorbide A, pheophorbide A analog, or pharmaceutically acceptable salt thereof is formulated into a pharmaceutical composition.
29. The use of any one of items 16 to 28, wherein the pheophorbide A, pheophorbide A analog, or pharmaceutically acceptable salt thereof is for intrapulmonary administration.
30. The use of any one of items 16 to 29, wherein the subject is a human.
31. The use of any one of items 16 to 29, wherein the subject is a non-human animal.
32. The use of item 31, wherein the non-human animal is a farm animal.
33. The use of item 31, wherein the non-human animal is a pet.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts an overlay of LC chromatogram (black line=PDA absorption at 410 nm), collected fractions (grey boxes), and assayed inhibition activity mapping strongest effects to well 34, containing Pheophorbide A, a major component of the botanical extracts.

FIGS. 2A-D show the results of LC-MS spiking experiments. FIG. 2A: UV-vis chromatogram (410 nm) showing chlorophyll derivatives in active botanical extract. FIG. 2B: UV-vis chromatogram (410 nm) of Pheophorbide A commercial reference standard. FIG. 2C: UV-vis chromatogram (410 nm) of active botanical extract spiked 1:1 with Pheophorbide A commercial reference standard. FIG. 2D: Differential display showing overlaid chromatograms (410 nm) normalized to Pheophorbide A peak intensity. Note: relative peak height of minor components in the botanical extract are reduced in intensity due to addition of Pheophorbide A.

FIGS. 3A and B depict UV-vis absorption spectra and extracted nominal mass ESI+ve MS traces of Pheophorbide A peak from active botanical extract (FIG. 3A) and reference standard (FIG. 3B). Both UV-vis absorption spectra are defined by maxima at 409 and 663 nm, while the nominal mass positive ion [M+H]⁺ of 593.3 m/z matches the predicted monotopic mass for [Pheophorbide A+H]⁺=593.275847 m/z.

FIG. 4 depicts a graph showing the dose dependent decrease in binding between SARS-CoV-2 spike protein an ACE2 in the presence of pheophorbide A (PPA), but not in the presence of a non-active molecule (cyanocobalamin).

FIGS. 5A-D are graphs showing that pheophorbide a (Pa) inhibits SARS-CoV-2 USA-WA1/2020 virus infection. SARS-CoV-2-permissive cell lines were infected with USA-WA1/2020 virus and then incubated with Pa. One hour later, Pa was photoactivated (Pact Pa) in culture or protected from light (Pa). Two days later, Huh-7.5 (FIG. 5A) and Vero E6 (FIG. 5B) cell viability was measured using the CellTiter-Glo® luminescent cell viability assay. Pa significantly inhibits SARS-CoV-2 USA-WA1/2020 virus variant infection in culture for both Huh-7.5 (FIG. 5C) and Vero E6 (FIG. 50) cell lines. Significance was determined using a two-way ANOVA compared to control (0 μM Pa), *P<0.05. Dashed line in FIGS. 5C-D is background absorbance (490 nm) for uninfected condition.

FIGS. 6A-B are graphs showing that pheophorbide a (Pa) inhibits SARS-CoV-2 alpha variant infection. SARS-CoV-2 alpha variant-infected Vero E6 cells were incubated with Pa. One hour later, Pa was photoactivated in culture (Pact Pa) or protected from light (Pa). Two days later, cell viability was measured using the CellTiter-Glo® luminescent cell viability assay (FIG. 6A). Pa significantly inhibits SARS-CoV-2 infection in culture and this inhibition is enhanced with photoactivation (FIG. 6B). Significance was determined using a two-way ANOVA compared to control (0 μM Pa), *P<0.05. Dashed line in FIG. 6B is background absorbance (490 nm) for uninfected condition.

FIGS. 7A-B are graphs showing that pheophorbide a (Pa) inhibits SARS-CoV-2 USA-WA1/2020 virus infection. SARS-CoV-2 USA-WA1/2020 virus-infected HT1080 ACE2+ cells were incubated with Pa. One hour later, Pa was photoactivated in culture (Pact Pa) or protected from light (Pa). Two days later, cell viability was measured using the CellTiter-Glo® luminescent cell viability assay (FIG. 7A). Pa significantly inhibits SARS-CoV-2 infection in culture (FIG. 7B). Significance was determined using a two-way ANOVA compared to control (0 μM Pa), *P<0.05. Dashed line in FIG. 7B is background absorbance (490 nm) for uninfected condition.

FIGS. 8A-B are graphs showing that pheophorbide a (Pa) inhibits SARS-CoV-2 alpha variant infection. SARS-CoV-2 alpha variant-infected A549 ACE2+ cells were incubated with Pa. One hour later, Pa was photoactivated in culture (Pact Pa) or protected from light (Pa). Two days later, cell viability was measured using the CellTiter-Glo® luminescent cell viability assay (FIG. 8A). Pa significantly inhibits SARS-CoV-2 infection in culture (FIG. 8B). Significance was determined using a two-way ANOVA compared to control (0 μM Pa), *P<0.05. Dashed line in FIG. 8B is background absorbance (490 nm) for uninfected condition.

FIGS. 9A-B are graphs showing that pheophorbide a (Pa) inhibits SARS-CoV-2 alpha variant infection. SARS-CoV-2 alpha variant-infected Huh-7.5 cells were incubated with Pa. One hour later, Pa was photoactivated in culture (Pact Pa) or protected from light (Pa). Two days later, cell viability was measured using the CellTiter-Glo® luminescent cell viability assay (FIG. 9A). Pa significantly inhibits SARS-CoV-2 infection in culture (FIG. 9B). Significance was determined using a two-way ANOVA compared to control (0 μM Pa), *P<0.05. Dashed line in FIG. 9B is background absorbance (490 nm) for uninfected condition.

FIGS. 10A-D are graphs showing that pheophorbide a (Pa) pre-treatment inhibits SARS-CoV-2 USA-WA1/2020 virus infection. SARS-CoV-2-permissive cell lines were incubated with Pa. One hour later, Pa was photoactivated in culture (Pact Pa) or protected from light (Pa) and subsequently infected with USA-WA1/2020 virus. Two days later, Huh-7.5 (FIG. 10A) and Vero E6 (FIG. 10B) cell viability was measured using the CellTiter-Glo® luminescent cell viability assay. Pa significantly inhibits SARS-CoV-2 USA-WA1/2020 virus variant infection in culture for both Huh-7.5 (FIG. 10C) and Vero E6 (FIG. 10D) cell lines. Significance was determined using a two-way ANOVA compared to control (0 μM Pa), *P<0.05. Dashed line in FIGS. 10C-D is background absorbance (490 nm) for uninfected condition.

FIGS. 11A-C show the structure of pheophorbide A and other compounds comprising a porphine ring tested in the studies described herein.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, virology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), J. E. Coligan et al. (editors) Current Protocols in Immunology, John Wiley & Sons (including all updates until present) and Current protocols in Microbiology, John Wiley & Sons (including all updates until present).

In the studies described herein, the present inventors have identified a small molecule inhibitor present in botanical extracts that blocks the interaction between SARS-CoV-2 spike protein and its receptor ACE2. This molecule is pheophorbide A, a naturally occurring compound produced in plants from chlorophyll A.

The present disclosure provides a method for blocking the entry and/or replication of a virus, for example a coronavirus such as SARS-CoV-2, in an ACE2-expressing cell, comprising contacting the cell with an effective amount of pheophorbide A, a pheophorbide A analog, or a pharmaceutically acceptable salt thereof. The present disclosure also provides the use of pheophorbide A, a pheophorbide A analog, or a pharmaceutically acceptable salt thereof for blocking the entry and/or replication of a virus, for example a coronavirus such as SARS-CoV-2, in an ACE2-expressing cell. The present disclosure also provides the use of pheophorbide A, a pheophorbide A analog, or a pharmaceutically acceptable salt thereof for the manufacture of a medicament for blocking the entry and/or replication of a virus, for example a coronavirus such as SARS-CoV-2, in an ACE2-expressing cell. The present disclosure also provides pheophorbide A, a pheophorbide A analog, or a pharmaceutically acceptable salt thereof for use in blocking the entry and/or replication of a virus, for example a coronavirus such as SARS-CoV-2, in an ACE2-expressing cell.

The present disclosure provides a method for treating an infection by a virus, preferably a coronavirus such as SARS-CoV-2, in a subject comprising administering to said subject an effective amount of pheophorbide A, a pheophorbide A analog, or a pharmaceutically acceptable salt thereof. The present disclosure also provides the use of pheophorbide A, a pheophorbide A analog, or a pharmaceutically acceptable salt thereof for treating an infection by a virus, for example a coronavirus such as SARS-CoV-2, in a subject. The present disclosure also provides the use of pheophorbide A, a pheophorbide A analog, or a pharmaceutically acceptable salt thereof for the manufacture of a medicament for treating an infection by a virus, preferably a coronavirus such as SARS-CoV-2, in a subject. The present disclosure also provides pheophorbide A, a pheophorbide A analog, or a pharmaceutically acceptable salt thereof for use in treating an infection by a virus, preferably a coronavirus such as SARS-CoV-2, in a subject.

The present disclosure provides a method for treating a viral disease, preferably a viral disease caused by coronavirus such as COVID-19, in a subject comprising administering to said subject an effective amount of pheophorbide A, a pheophorbide A analog, or a pharmaceutically acceptable salt thereof. The present disclosure also provides the use of pheophorbide A, a pheophorbide A analog, or a pharmaceutically acceptable salt thereof for treating a viral disease, preferably a viral disease caused by coronavirus such as COVID-19, in a subject. The present disclosure also provides the use of pheophorbide A, a pheophorbide A analog, or a pharmaceutically acceptable salt thereof for the manufacture of a medicament for treating a viral disease, preferably a viral disease caused by coronavirus such as COVID-19, in a subject. The present disclosure also provides pheophorbide A, a pheophorbide A analog, or a pharmaceutically acceptable salt thereof for use in treating a viral disease, preferably a viral disease caused by coronavirus such as COVID-19, in a subject.

The present disclosure provides a method for reducing the risk of developing a coronavirus-related disease such as COVID-19, or the severity of a coronavirus-related disease (e.g., COVID-19) in a subject, the method comprising administering to said subject an effective amount of pheophorbide A, a pheophorbide A analog, or a pharmaceutically acceptable salt thereof. The present disclosure also provides the use of pheophorbide A, a pheophorbide A analog, or a pharmaceutically acceptable salt thereof for reducing the risk of developing and/or the severity of a coronavirus-related disease such as COVID-19 in a subject. The present disclosure also provides the use of pheophorbide A, a pheophorbide A analog, or a pharmaceutically acceptable salt thereof for the manufacture of a medicament for reducing the risk of developing a coronavirus-related disease such as COVID-19, or the severity of a coronavirus-related disease (e.g., COVID-19) in a subject. The present disclosure also provides pheophorbide A, a pheophorbide A analog, or a pharmaceutically acceptable salt thereof for use in reducing the risk of developing a coronavirus-related disease such as COVID-19, or the severity of a coronavirus-related disease (e.g., COVID-19) in a subject.

Pheophorbide A (PPBa) is the dephytylation and demetallation product of chlorophyll a, which is formed in algae and higher plants.

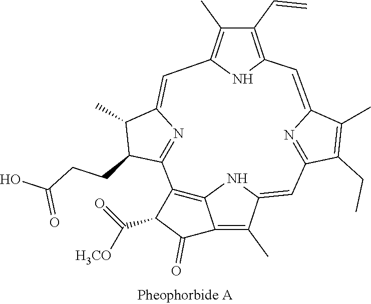 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)O)[C@@H]3C]

PPBa may be found or isolated from a variety of sources including plant and algae extracts such as extracts from Dunaliella primolecta, Cylindrotheca closterium, Morinda citrifolia, Enteromorpha prolifera, Spinacia oleracea (spinach), Spirulina, Mentha piperita, Arrabidaea chica, Gelidium amansii, Scutellaria barbata (see, e.g., Saide et al., Mar. Drugs 2020, 18(5), 257; CN103031354A; Kobayashi et al., Bioscience, Biotechnology, and Biochemistry, Volume 83, 2019—Issue 7, Miranda et al., Photodiagnosis and Photodynamic Therapy, 3 Jun. 2017, 19:256-265; WO 2017/086536; Tang et al., Cancer Biology & Therapy, 5:9, 1111-1116).

In an embodiment, the extract is a Clintonia borealis L. extract (leaves), a Lupinus polyphyllus L. extract (leaves), a Caltha palustris L. extract (leaves), a Quercus macrocarpa L. extract (leaves), a Prunus americana L. extract (flower) or a Caragana arborescens L. extract (bark).

Examples of pheophorbide A analogs include pheophorbide-A methyl ester, (R,S)-13(2)-hydroxypheophorbide-A methyl ester, 15(2)-hydroxylactone pheophorbide-A methyl ester and 15(2)-methoxylactone pheophorbide-A methyl ester (see, e.g., Kamarulzaman et al., Chem Biodivers 2011 March; 8(3):494-502).

Pheophorbide A may also be in the form of a pharmaceutically acceptable salt, such as HCl—N-Pheophorbide A. The term “pharmaceutically acceptable salts” means salts of pheophorbide A which retain pharmacological activities of interest, i.e., activities to block the entry of SARS-CoV-2 in an ACE2-expressing cell. These salts may be formed using inorganic acids such as hydrochloride, hydrobromide and hydroiodide, or organic acids such as acetate, adipate, alginate, aspartate, benzoate, benzenesulfonate, p-toluenesulfonate, bisulfate, sulfamate, sulfate, naphthylate, butyrate, citrate, camphorate, camphosulfate, cyclopentanepropionate, digluconate, dodecylsulfate, ethanesulfate, fumarate, glucoheptanoate, glycerophosphate, hemisulfate, heptanoate, hexanoate, 2-hydroxyethanesulfate, lactate, maleate, methanesulfonate, 2-naphthalenesulfonate, nicotinate, oxalate, tosylate and undecanoate.

Pheophorbide A may be complexed to a metal, such as magnesium (main form found in nature), copper, nickel, cobalt, zinc or sodium. In an embodiment, the pheophorbide A is not in the form of zinc pheophorbide A (ZnPh).

For the method, use, and therapy described herein, the pheophorbide A, a pheophorbide A analog, or a pharmaceutically acceptable salt thereof may be used in the form of an extract (e.g., plant or algae extract) comprising a suitable amount of pheophorbide A, a pheophorbide A analog, or a pharmaceutically acceptable salt thereof, including a crude extract or a partially purified extract enriched in pheophorbide A, a pheophorbide A analog, or a pharmaceutically acceptable salt thereof, or may be in purified form (either isolated from a natural source such as a plant or algae or synthesized). Thus, in an embodiment, an extract comprising pheophorbide A, a pheophorbide A analog, or a pharmaceutically acceptable salt thereof is used or administered. In another embodiment, purified or isolated pheophorbide A, a pheophorbide A analog, or a pharmaceutically acceptable salt thereof is used or administered.

As used herein, the term “isolated” or “purified” means that the pheophorbide A, pheophorbide A analog, or a pharmaceutically acceptable salt thereof is in a physical form that is non-identical to the form found in nature (e.g., at a purity not found in nature). “isolated” or “purified” does not require, although it does not prohibit, that the pheophorbide A analog, or a pharmaceutically acceptable salt thereof be 100% pure. In an embodiment, the purified or isolated pheophorbide A analog, or a pharmaceutically acceptable salt thereof represents at least 10% of the total components present in an extract or a preparation. In preferred embodiments, the purified or isolated pheophorbide A analog, or a pharmaceutically acceptable salt thereof represents at least about 20%, 30%, 40%, 50%, 60%, 70%, 80%, 85%, 90% or 95% of the total components present in an extract or a preparation.

In an embodiment, the methods or uses described herein do not include photodynamic therapy (PDT), i.e., the pheophorbide A, pheophorbide A analog, or a pharmaceutically acceptable salt thereof is not used in combination with PDT.

In another embodiment, the pheophorbide A, pheophorbide A analog, or a pharmaceutically acceptable salt thereof is photoactivated. In an embodiment, the methods and uses described herein include contacting the pheophorbide A, pheophorbide A analog, or a pharmaceutically acceptable salt thereof with light, i.e., illuminating the pheophorbide A, pheophorbide A analog, or a pharmaceutically acceptable salt thereof with excitation light. In an embodiment, the methods or uses described herein are combined with photodynamic therapy (PDT). The pheophorbide A, pheophorbide A analog, or a pharmaceutically acceptable salt thereof may be contacted with any light of a proper wavelength (e.g., wavelengths comprised between 450 nm and 595 nm) and sufficient power to activate the pheophorbide A, pheophorbide A analog, or a pharmaceutically acceptable salt thereof, such as a light-emitting diode (LED).

In another embodiment, the methods or uses described hereon do not include zinc supplementation, i.e., the pheophorbide A, pheophorbide A analog, or a pharmaceutically acceptable salt thereof is not used in combination with zinc supplementation.

The skilled person would understand that the extract or the purified pheophorbide A, a pheophorbide A analog, or a pharmaceutically acceptable salt thereof may be mixed with one or more carriers and/or excipients (pharmaceutically acceptable carriers and/or excipients) to obtain a composition suitable for administration to the subject.

An “excipient,” as used herein, has its normal meaning in the art and is any ingredient that is not an active ingredient (drug) itself. Excipients include for example buffers, binders, lubricants, diluents, fillers, thickening agents, disintegrants, plasticizers, coatings, barrier layer formulations, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol, nebulizer) administration. Therapeutic compositions are prepared using standard methods known in the art by mixing the active ingredient having the desired degree of purity with one or more optional pharmaceutically acceptable carriers, excipients and/or stabilizers (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press).

In an embodiment, the pheophorbide A, a pheophorbide A analog, or a pharmaceutically acceptable salt thereof is formulated for oral administration. Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In an embodiment, the pheophorbide A, a pheophorbide A analog, or a pharmaceutically acceptable salt thereof is formulated for parenteral administration (e.g., injection). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylenevinyl acetate copolymer particles, osmotic pumps, implantable infusion systems, and liposomes.

Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

In an embodiment, the pheophorbide A, a pheophorbide A analog, or a pharmaceutically acceptable salt thereof is formulated for administration into the respiratory tract. In an embodiment, the pheophorbide A, a pheophorbide A analog, or a pharmaceutically acceptable salt thereof is formulated for pulmonary administration, e.g., in the form of an aerosol or spray. The pheophorbide A, a pheophorbide A analog, or a pharmaceutically acceptable salt thereof may be administered using an inhalation device such as a metered-dose inhaler, dry powder inhaler or a nebulizer. Formulation for pulmonary administration typically include excipients such as sugars/polysaccharides, polymers, amino acids, viscosity modifiers, surfactants, propellants, lipids (e.g., to form liposomes), etc. Formulation for pulmonary administration may be in unit-dose or multidose presentations. In another aspect, the present disclosure provides an inhalation device comprising pheophorbide A, a pheophorbide A analog, or a pharmaceutically acceptable salt thereof, or a composition comprising same. In another embodiment, the pheophorbide A, a pheophorbide A analog, or a pharmaceutically acceptable salt thereof is formulated for nasal administration, e.g., in the form of a nasal spray. In another embodiment, the pheophorbide A, a pheophorbide A analog, or a pharmaceutically acceptable salt thereof is formulated in the form of a mouth rinse.

For the prevention, treatment or reduction in the severity of a given disease or condition (viral disease such as COVID-19), the appropriate dosage of pheophorbide A, a pheophorbide A analog, or a pharmaceutically acceptable salt thereof will depend on the type of disease or condition to be treated, the severity and course of the disease or condition, whether the pheophorbide A, a pheophorbide A analog, or a pharmaceutically acceptable salt thereof is administered for preventive or therapeutic purposes, previous therapy, the patient's clinical history and response to the pheophorbide A, a pheophorbide A analog, or a pharmaceutically acceptable salt thereof, and the discretion of the attending physician. The pheophorbide A, a pheophorbide A analog, or a pharmaceutically acceptable salt thereof may be suitably administered to the patient at one time or over a series of treatments. Preferably, it is desirable to determine the dose-response curve in vitro, and then in useful animal models prior to testing in humans. The present disclosure provides dosages for the pheophorbide A, a pheophorbide A analog, or a pharmaceutically acceptable salt thereof and compositions comprising same. For example, depending on the type and severity of the disease, about 1 μg/kg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

As used herein the term “treating” or “treatment” in reference to viral disease is meant to refer to a reduction/improvement in one or more symptoms or pathological features associated with said viral disease (e.g., COVID-19), such as a reduction of the occurrence and/or severity of one or more symptoms. Non-limiting examples include a decrease in viral load, reduction of cough, fever, fatigue, shortness of breath, reduction/prevention of acute respiratory distress syndrome (ARDS), reduction/prevention of multi-organ failure, septic shock, blood clots, hospitalization, need for ICU involving intubation, etc.

In an embodiment, the methods and uses defined herein are for the prevention, treatment and/or management of infections by the Wuhan original SARS-CoV-2 variant. In another embodiment, the methods and uses defined herein are for the prevention, treatment and/or management of infections by any variants of the Wuhan original SARS-CoV-2 variant. Examples of such variants include the B.1.1.7 (also known as VOC-202012/01 or alpha (a)), 501Y.V2 (also known as B.1.351 or beta (p)), P.1 (also known as B.1.1.28.1 or gamma (γ)), B.1.617.2 (also known as delta (6)), or B.1.1.529 (omicron) variant, as well as other variants of concern (VOC) such as B.1.429, B.1.526, B.1.525, and A.23.1 (see, e.g., www.cdc.gov/coronavirus/2019-ncov/cases-updates/variant-surveillance/variant-info.html). In an embodiment, the methods and uses defined herein are for the prevention, treatment and/or management of infections by the SARS-CoV-2 Delta (6) variant. In an embodiment, the methods and uses defined herein are for the prevention, treatment and/or management of infections by the SARS-CoV-2 Omicron variant, e.g., Omicron sublineage BA.1, BA.2 and/or BA.3. In an embodiment, the methods and uses defined herein are for the prevention, treatment and/or management of infections by any new SARS-CoV-2 variants.

The pheophorbide A, a pheophorbide A analog, or a pharmaceutically acceptable salt thereof may be used alone or in combination with other prophylactic or therapeutic agents such as antivirals, anti-inflammatory agents, vaccines, immunotherapies, etc. The combination of active agents and/or compositions comprising same may be administered or co-administered (e.g., consecutively, simultaneously, at different times) in any conventional dosage form. Co-administration in the context of the present disclosure refers to the administration of more than one therapeutic throughout the course of a coordinated treatment to achieve an improved clinical outcome. Such co-administration may also be coextensive, that is, occurring during overlapping periods of time. For example, a first agent (e.g., the pheophorbide A, a pheophorbide A analog, or a pharmaceutically acceptable salt thereof) may be administered to a patient before, concomitantly, before and after, or after a second active agent (e.g., an antiviral or anti-inflammatory agent) is administered. The agents may in an embodiment be combined/formulated in a single composition and thus administered at the same time.

In an embodiment, the pheophorbide A, a pheophorbide A analog, or a pharmaceutically acceptable salt thereof is for administration prior to development of the viral disease (e.g., COVID-19). In another embodiment, the pheophorbide A, a pheophorbide A analog, or a pharmaceutically acceptable salt thereof is for administration after development of the viral disease (e.g., COVID-19). In another embodiment, the pheophorbide A, a pheophorbide A analog, or a pharmaceutically acceptable salt thereof is for administration prior to and after development of the viral disease (e.g., COVID-19).

As used herein, the term “subject” is taken to mean warm blooded animals such as mammals, for example, cats, dogs, mice, guinea pigs, horses, bovine cows, sheep, non-human primates and humans. In an embodiment, the subject is a mammal, and more particularly a human.

In an embodiment, the subject or patient has a weakened immune system and a reduced ability to fight viral infections such as SARS-CoV-2 infection. In another embodiment, the subject or patient is an immunosuppressed or immunocompromised subject or patient.

Immunosuppression may be caused by certain diseases or conditions, such as AIDS, cancer, diabetes, malnutrition, and certain genetic disorders, or certain drugs or treatments such as anticancer drugs, radiation therapy, and stem cell or organ transplant. In an embodiment, the subject or patient is an elderly subject or patient, for example a subject or patient having 60 years old or more, 65 years old or more, 70 years old or more, 75 years old or more, or 80 years old or more, who typically develop a weaker immune response to vaccines and infections.

### EXAMPLES

The present disclosure is illustrated in further details by the following non-limiting examples.

### Example 1: Screening of Botanical Extracts

A library containing 150 dried botanical samples was collected. One gram of each dried botanical material (leaves, flowers, fruits, bark, and roots) was extracted using mechanical disruption by Polytron immersion homogenizer in 30 mL of an 3:1 acetonitrile:water (1% formic acid) solvent mixture. Samples were incubated at 35° C. for one hour in a sonic bath and then QuEChERs salts (AOAC method 2007.1; 6.0 g MgSO₄+1.5 g NaOAc) were added to promote phase separation into aqueous and organic phases. After vortexing and centrifugation, 20 mL aliquots of the organic phase were dried under a nitrogen gas stream, and dissolved in ethanol at a concentration of 20 mg/mL.

The crude extracts were screened using a SARS-CoV-2 Surrogate Virus Neutralization Test (sVNT) in which the ACE2 target protein was coated onto the solid phase of 96-well polystyrene ELISA plates and the detection was a fusion protein of the SARS-CoV-2 spike protein S1 subunit and the horseradish peroxidase (HRP) enzyme. The extract samples (20 mg/mL) were diluted in assay buffer (PBS/BSA) to a final concentration of 5 mg/mL. 40 μL of the extract sample was combined with 60 μL of the detection fusion protein in assay buffer. SARS-CoV-2 neutralizing monoclonal antibodies were used as positive controls. Ethanol in assay buffer at the same concentration as the extract samples were used as negative controls.

Extracts 23, 14, and 20 showed inhibition comparable to >90% of that of the positive control neutralizing antibody while samples 124, 32, and 86 showed >88% activity of the control neutralizing antibody. All other samples showed low level inhibition in the surrogate virus neutralization test.

TABLE 1
Crude extracts with significant biological activity.

| Sample | | Percent |
|---|---|---|
| number | Material | Inhibition |
| 23 | Blue Bead Lily (Clintonia borealis L.) | 95.80% |
| | leaves, lyophilized | |
| 14 | Big Leaf Lupine (Lupinus polyphyllus L.) | 94.40% |
| | leaves, lyophilized | |
| 20 | Marsh Marigold (Caltha palustris L.) | 92.00% |
| | leaves, lyophilized | |
| 124 | Burr Oak (Quercus macrocarpa L.) | 89.90% |
| | leaves, lyophilized | |
| 32 | American plum (Prunus americana L.) | 88.30% |
| | flower, lyophilized | |
| 86 | Siberian Peashrub (Caragana arborescens L.) | 88.30% |
| | bark, lyophilized | |

All of the crude extracts were subsequently screened against the detection enzyme component of the assay by coating the enzyme directly on the plate to verify that the observed inhibition was a result of a blocked interaction between the SARS-CoV-2 spike and ACE2 proteins, and not simply a small molecule inhibitor of the enzyme reporter conjugated to SARS-CoV-2. Briefly, the reporter enzyme, HRP, was coated onto 96-well ELISA plates and blocked using BSA-PBS. After washing the plates, the crude botanical extracts were applied to the coated plates and incubated for 1 hour at room temperature with shaking at 300 RPM. The plates were washed three times with PBS containing 0.05% Tween™ 20, and then incubated with the colorimetric substrate. None of the extracts had activity directly against the reporter enzyme, indicating that the inhibition observed in the sVNT was due to a molecule actively blocking the interaction between the SARS-CoV-2 spike and ACE2 proteins.

### Example 2: Identification of the Active Compound in the Crude Extracts with Significant Biological Activity

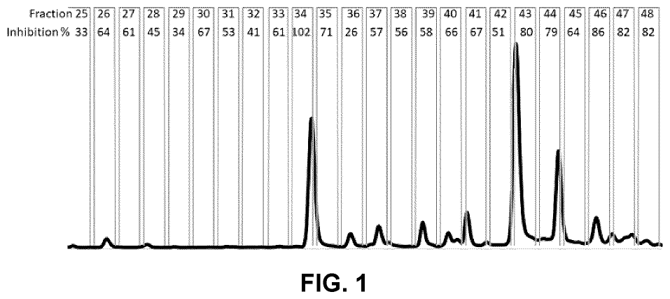

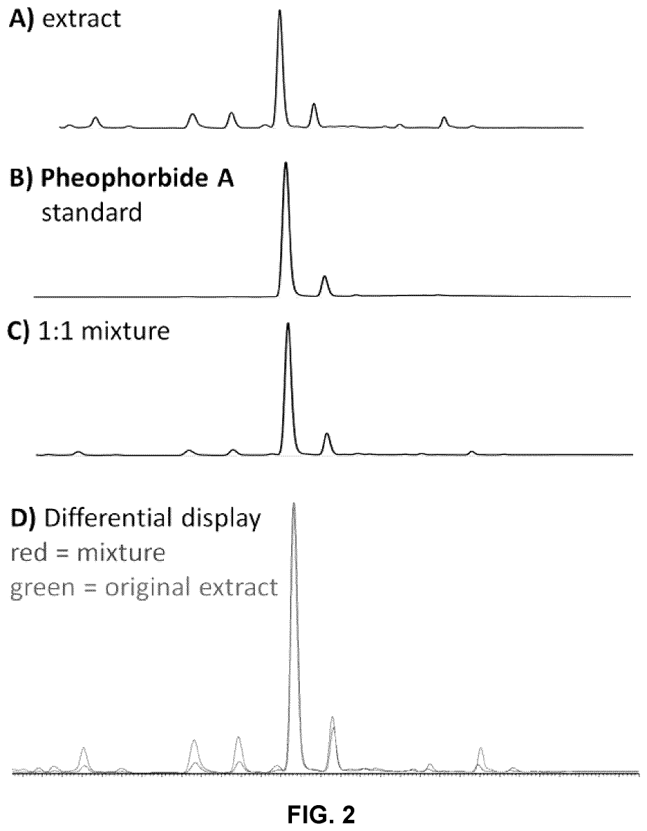

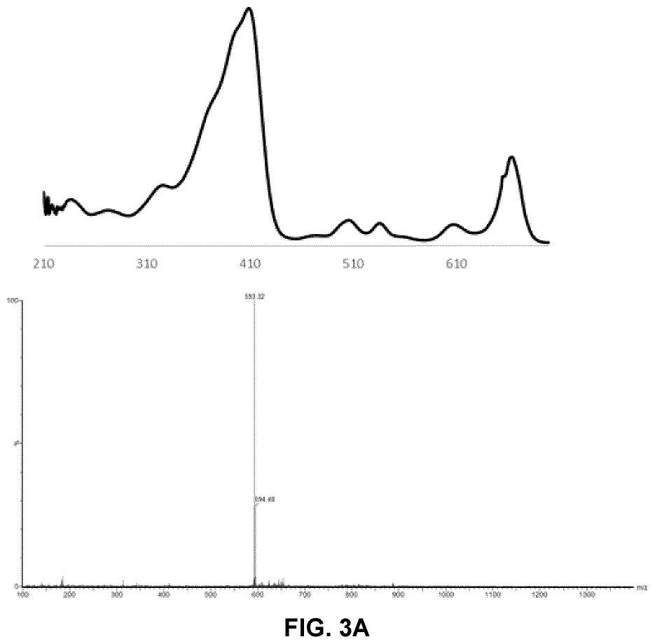

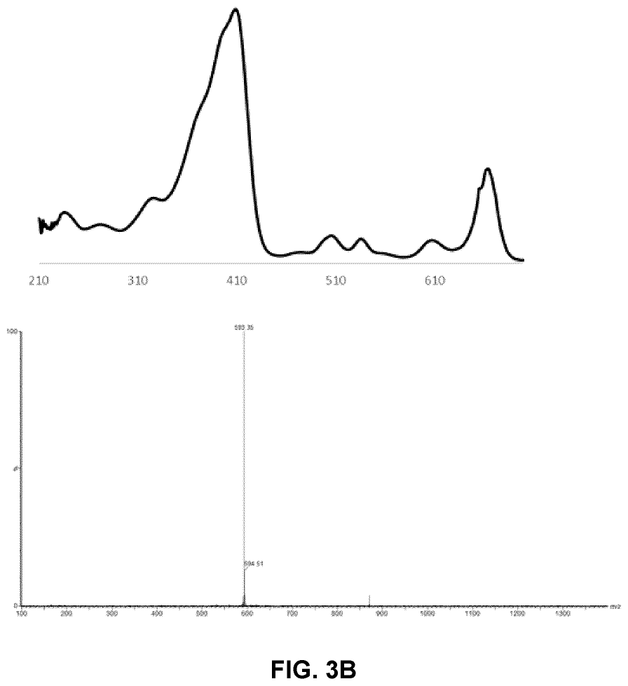

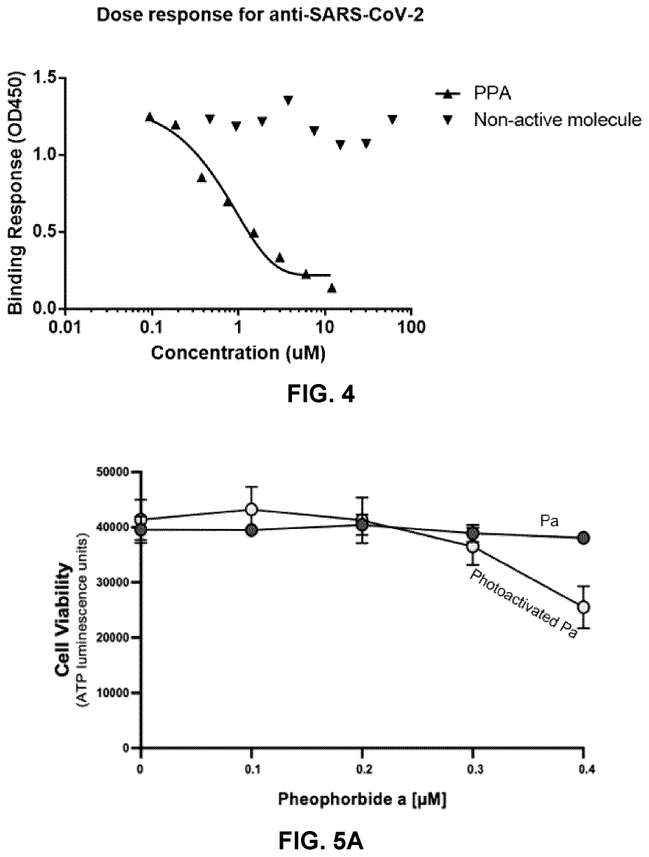

The six crude extracts with significant biological activity were selected for further fractionation and characterization by liquid chromatography/mass spectrometry (LC/MS). For these six lead active extracts, 200 μg of each were separated via reverse phase (RP)-C18 LC/MS using water:acetonitrile gradients into 8×0.500 mL sub-fractions by a high precision robotic fraction collector. The fractions were dried down for subsequent inhibition screening as described above. The components in the active fractions were identified by LC/MS using UV-visible absorption data, m/z ratios, and congruent retention times with commercial standards. All six extracts possessed inhibitory activity which mapped to the same fractions which were found to contain a simple mixture of chlorophyll derivatives, whereas inactive crude extracts lacked these peaks. Subsequent RP-C18 fractionation of one lead extract (Sample number 14—Big Leaf Lupine) into 62.5 μL sub-fractions following an optimized chromatographic gradient showed the strongest inhibitory activity was associated with fraction #34, containing one main peak (FIG. 1). Further LC-MS spiking experiments performed using the botanical extract alone, commercial standard alone, and in a 1:1 mixed combination demonstrated that the active peak in fraction #34 corresponds to the chlorophyll derivative pheophorbide A (FIGS. 2A-D and 3A-B). To confirm that pheophorbide A is the compound conferring the SARS-CoV-2 inhibition activity measured in the extracts, pure pheophorbide A obtained from a commercial supplier was tested in the sVNT assay described at Example 1. As shown in FIG. 4, a dose-dependent inhibition of the binding of SARS-CoV-2 Spike protein to ACE2 was observed in the presence of pheophorbide A (PPA).

### Example 3: Testing of Other Structurally Related Compounds

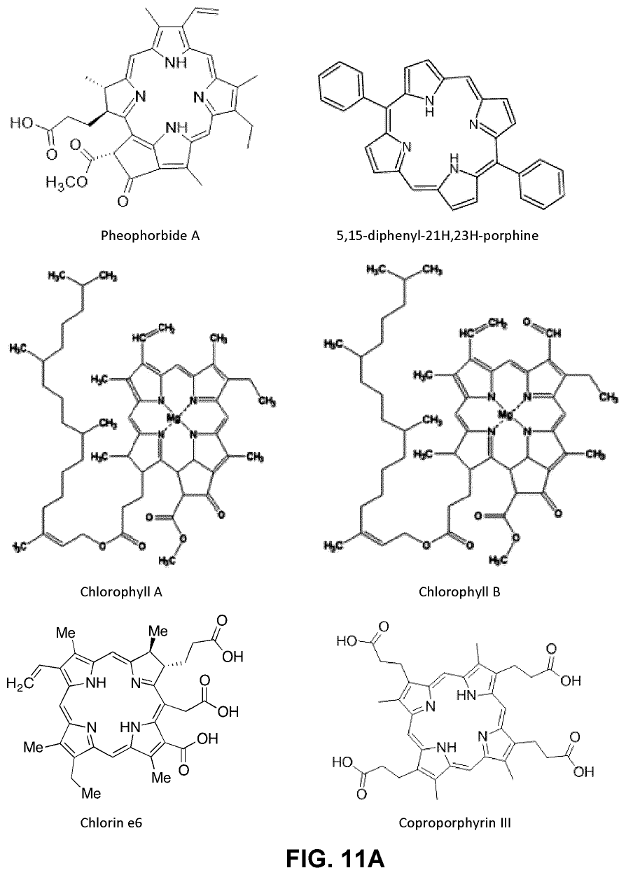

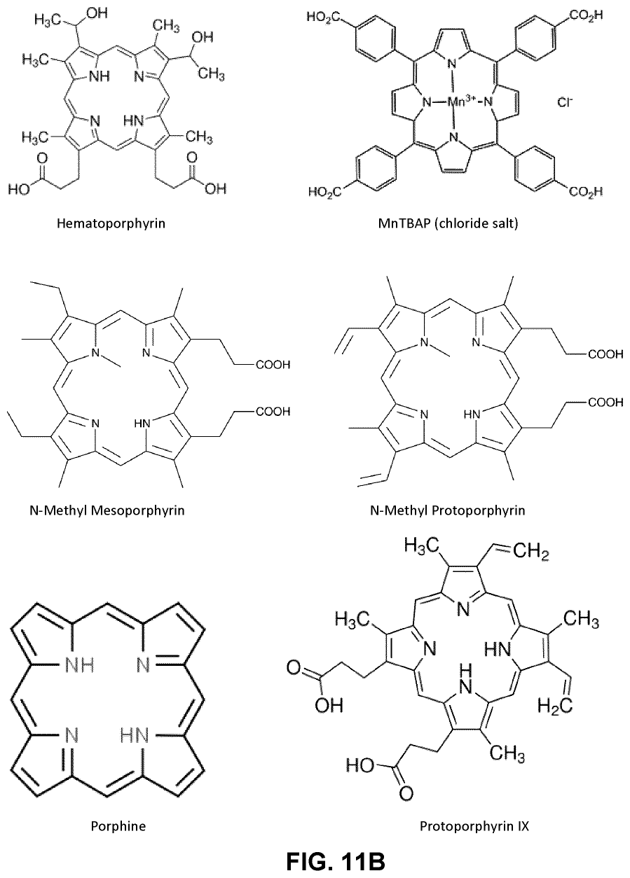

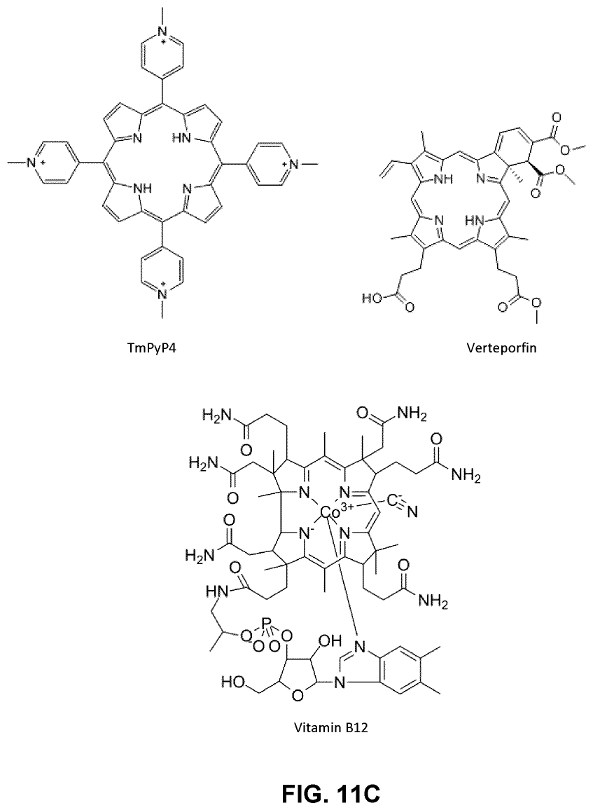

Other commercially available compounds structurally related to pheophorbide A based on the presence of a porphine ring (FIGS. 11A-C) were tested for their ability to inhibit the binding of SARS-CoV-2 Spike protein to ACE2 using the sVNT assay. The results reported in Table 2 show that most of the compounds tested do not inhibit of the binding of SARS-CoV-2 Spike protein to ACE2. Only two non-natural compounds, Protoporphyrin IX and Verteporfin, were found to have similar inhibitory activity.

TABLE 2
Activity of commercially available compounds
structurally related to pheophorbide A

| Compounds | Active (Y or N) | IC₅₀ | |
|---|---|---|---|
| Pheophorbide A | Y | 500 | nM |
| 5,15-diphenyl-21H,23H-porphine | N | N/A | |
| Chlorophyll A | N | N/A | |
| Chlorophyll B | N | N/A | |
| Chlorin E6 | Y (weak) | 5000 | nM |
| Coproporphyrin III | N | N/A | |
| Hematoporphyrin | N | N/A | |
| MnTBAP | N | N/A | |
| N-Methyl Mesoporphyrin | N | N/A | |
| N-Methyl Protoporphyrin | N | N/A | |
| Porphine | N | N/A | |
| Protoporphyrin IX | Y | 1200 | nM |
| Temoporfin | N | N/A | |
| TmPyP4 | N | N/A | |
| Verteporfin | Y | 225 | nM |

| | | |
|---|---|---|
| Vitamin B12 | N | N/A |

The results depicted in Table 2 show that the presence of a porphine ring is not sufficient to confer the ability to inhibit the binding of SARS-CoV-2 Spike protein to ACE2.

### Example 4: Effect of Pheophorbide a on SARS-CoV-2 Infection

### Methods

Replication-competent SARS-CoV-2 infection. One day prior to infection, 2×10⁴ cells from SARS-CoV-2 permissive cell lines (Vero E6, Huh-7.5, A549 ACE2+ and HT1080 ACE2+ cell lines) were seeded per well of a quadruplicate 96 well flat bottom plates and incubated overnight (37° C./5% CO₂). On the day of infection and in a Biosafety Level 3 laboratory (ImPaKT Facility, Western University), 10³ tissue culture medium infectious dose/milliliter (TCID₅₀/mL) SARS-CoV-2 replication-competent virus was prepared in minimum essential media (MEM)+2% fetal bovine serum (FBS), added to each respective well at a volume corresponding to 500 TCID₅₀ per well and incubated for one hour at 37° C. Depending on the experiment, SARS-CoV-2 USA/WA1/2020 virus or alpha variant (B.1.1.7 lineage) was used for infection. Virus inoculum was then aspirated and replaced with MEM+2% FBS containing a pre-determined concentration of pheophorbide a (Pa) or vehicle control. Following a further one-hour incubation at 37° C., media was aspirated and wells were washed twice with pre-warmed phosphate buffered saline (PBS). Pre-warmed MEM+2% FBS was added at 100 μL per well and two plates were subject to photoactivation for 15 minutes while the remaining two plates were protected from light during this period. In a subset of experiments, Pa addition to culture and SARS-CoV-2 infection were performed exactly as above; however, cell monolayers were pre-treated with Pa (±photoactivation) and then infected with SARS-CoV-2 USA-WA1/2020. Regardless of experimental subset, all plates were then incubated for 48 hours (37° C./5% CO₂). Two days later, one photoactivated plate and one plate protected from light were then use for either viability assessment (A.2) or to assess SARS-CoV-2 infection (A.3) as described below.

CellTiter-Glo® luminescent cell viability assay. One plate corresponding to each condition described above (±photoactivation) was removed from the incubator and left for 30 minutes at room temperature in the dark to equilibrate alongside CellTiter-Glo® reagent (Promega). An amount of CellTiter-Glo® reagent equivalent to the volume of media in each well was added per well and then mixed to ensure sufficient cell lysis. Following a 10 minute incubation, 100 μL from each well was transferred to its corresponding well on an opaque black 96 well plate and the luminescence was subsequently measured using a Synergy LX multi-mode reader and Gen5™ microplate reader and imager software (BioTek®) with a 1 second integration time and quantified as ATP luminescent units.

Microneutralization assay. A previously described microneutralization assay (Gasser R et al. Major role of IgM in the neutralizing activity of convalescent plasma against SARS-CoV-2. Cell Rep 2021; 34; 108790) was performed to quantify live SARS-CoV-2 infection. Briefly, media was discarded from the remaining two 96 well plates and monolayers were cross-linked with 10% formaldehyde for 24 hours. Wells were washed with PBS, permeabilized for 15 minutes with PBS+0.1% Triton X-100 (BDH Laboratory Reagents), washed with PBS again and then incubated for one hour at room temperature with PBS+3% non-fat milk. An anti-mouse SARS-CoV-2 nucleocapsid protein primary antibody solution (1 μg/mL, clone 1C7, Bioss Antibodies) was prepared in PBS+1% non-fat milk and added to all wells for one hour at room temperature.

Following extensive washing with PBS, an anti-mouse IgG horse radish peroxidase (HRP) secondary antibody solution was formulated in PBS+1% non-fat milk. One hour post incubation, wells were washed with PBS, SIGMAFAST™ o-phenylenediamine dihydrochloride (OPD) developing solution (Millipore Sigma) was prepared as per manufacturer's instructions and added to each well for 12 minutes. Dilute hydrogen chloride (HCl) at 3.0 M was added to quench the reaction and the optical density at 490 nm of the culture plates was immediately measured using a Synergy LX multi-mode reader and Gen5™ microplate reader and imager software (BioTek®).

### Results

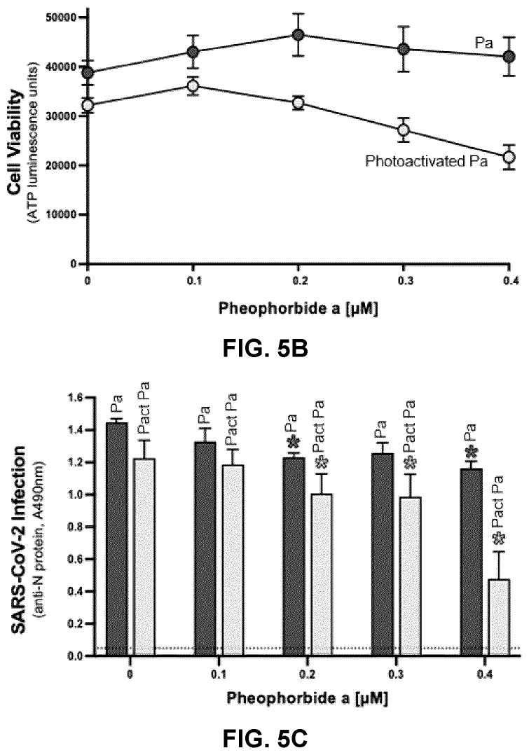

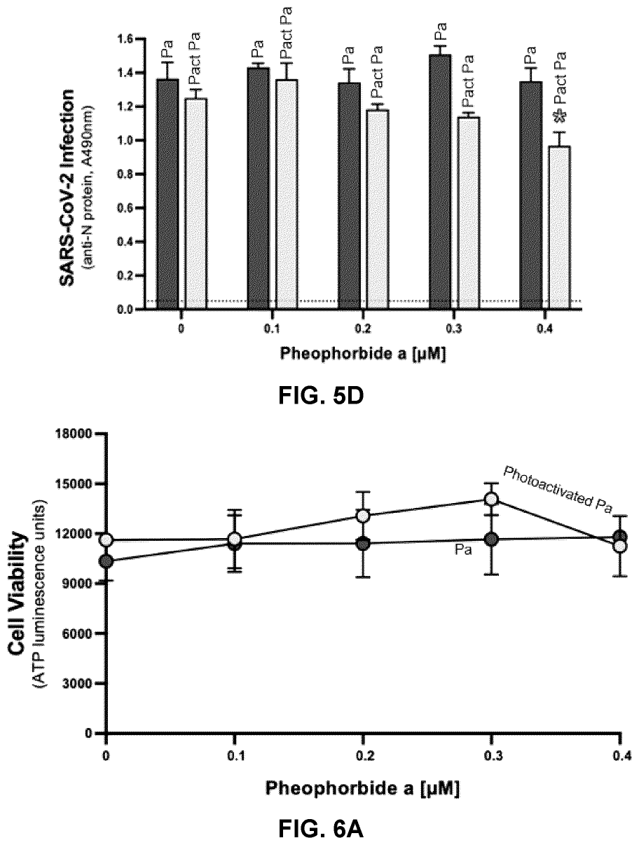

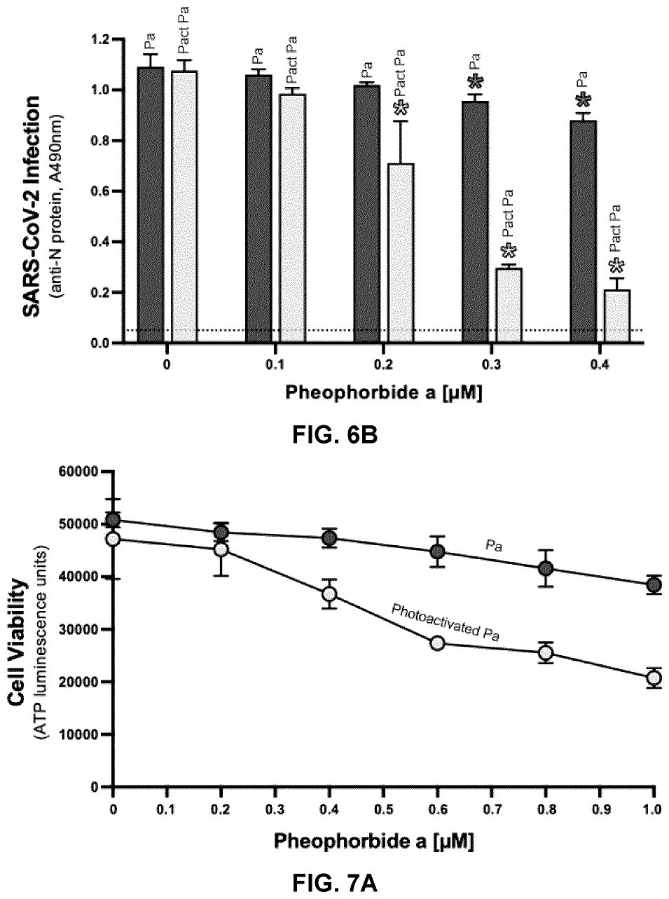

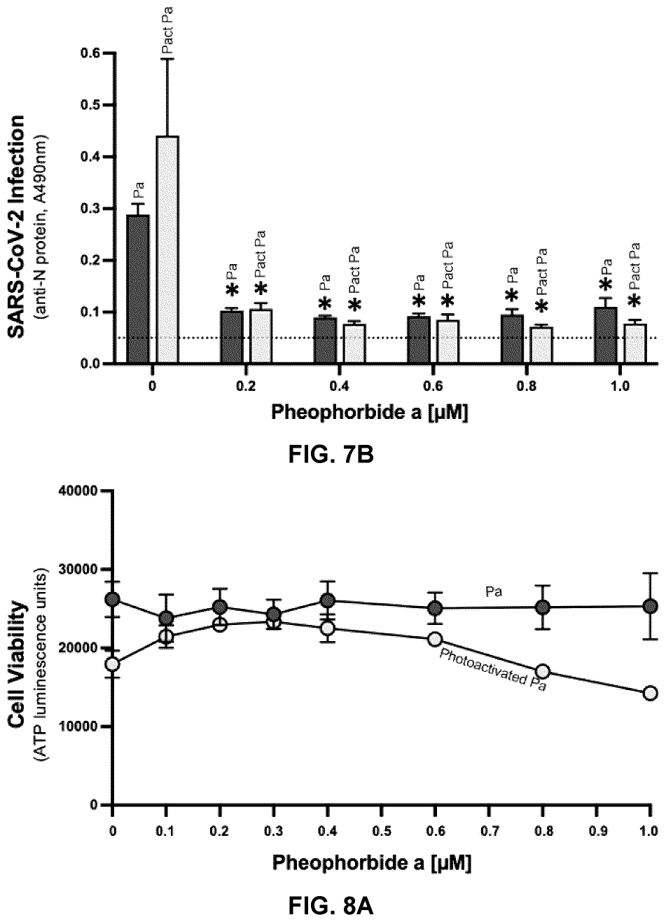

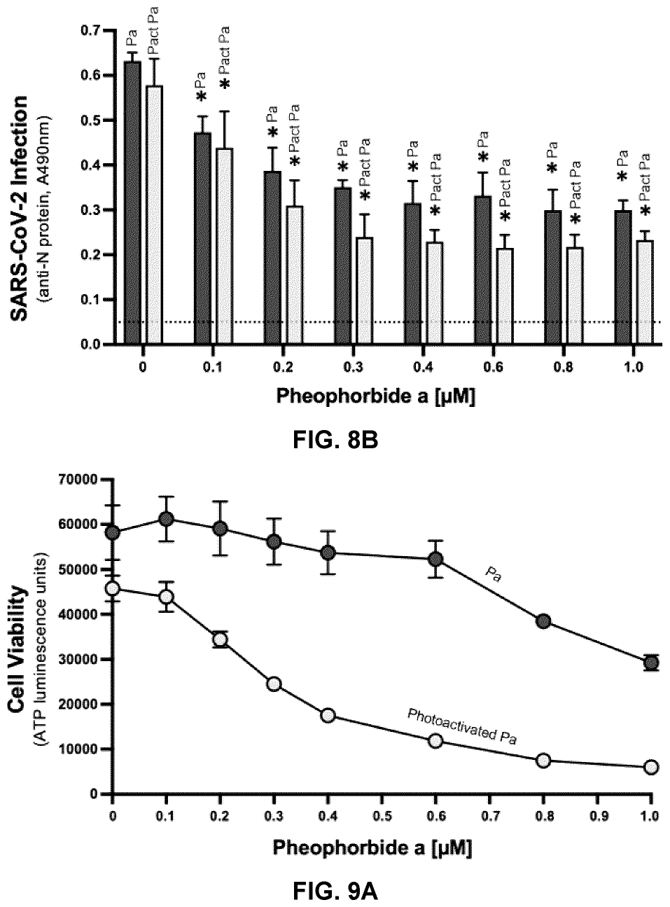

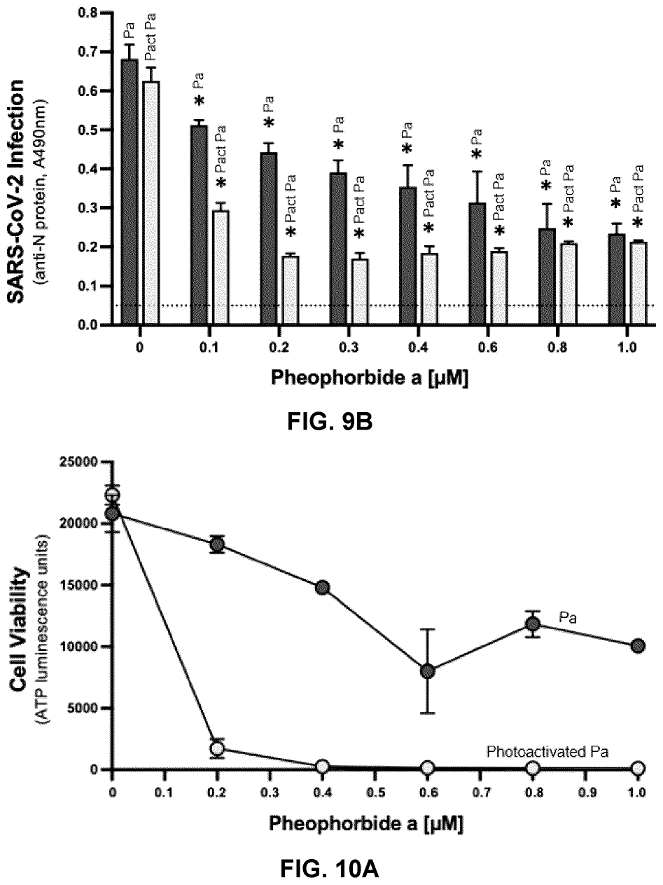

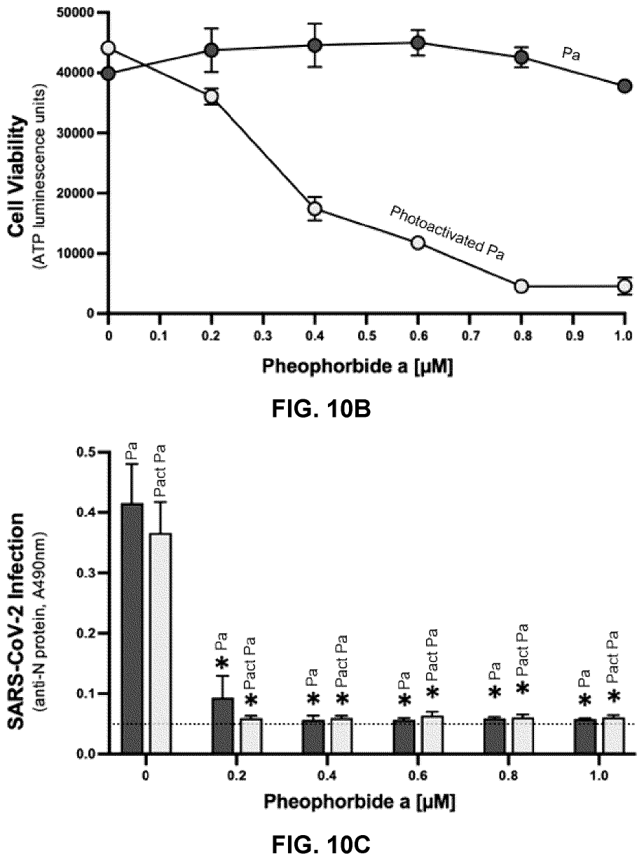

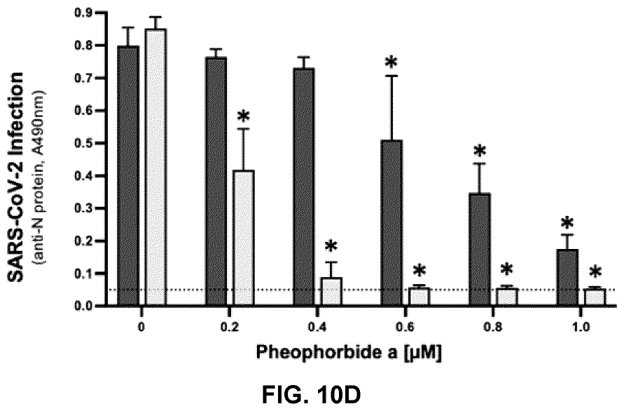

The results presented in FIGS. 5-10 show that pheophorbide A inhibits SARS-CoV-2 infection in a dose-dependent manner, and the antiviral activity was detected at doses lower than the dosing where pheophorbide A was toxic to the infected cells. This effect was observed in different SARS-CoV-2-permissive cell types (Huh-7.5 human hepatoma cell line, Vero E6 kidney epithelial cell line, ACE2⁺ HT-1080 fibrosarcoma cell line and ACE2⁺ A549 human alveolar basal epithelial cell line) and using different SARS-CoV-2 variants (USA-WA1/2020 and alpha variant). Pheophorbide A was shown to exhibit antiviral activity in the dark (i.e., without photoactivation), but the activity was generally improved under light exposure (photoactivation). The antiviral effect was more pronounced when the cells were pre-treated with pheophorbide A prior to SARS-CoV-2 exposure (FIGS. 10A-D).

Overall, the studies reported herein provide compelling evidence that the naturally-occurring chlorophyll derivative pheophorbide A, which is found in abundance in some plant species and has low toxicity in humans, may be useful for the prevention and/or treatment of SARS-CoV-2 infection and COVID-19.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method for blocking the entry and/or replication of severe acute respiratory syndrome coronavirus-2 (SARS-CoV-2) in an ACE2-expressing cell, the method comprising contacting the cell with an effective amount of pheophorbide A, a pheophorbide A analog, or a pharmaceutically acceptable salt thereof.

2. A method for treating an infection by SARS-CoV-2 in a subject comprising administering to said subject an effective amount of pheophorbide A, a pheophorbide A analog, or a pharmaceutically acceptable salt thereof.

3. A method for preventing or treating COVID-19 in a subject comprising administering to said subject an effective amount of pheophorbide A, a pheophorbide A analog, or a pharmaceutically acceptable salt thereof.

4. A method for reducing the risk of developing COVID-19 or the severity of COVID-19 in a subject, the method comprising administering to said subject an effective amount of pheophorbide A, a pheophorbide A analog, or a pharmaceutically acceptable salt thereof.

5. The method of claim 1, wherein an effective amount of pheophorbide A is administered.

6. The method of claim 1, wherein the pheophorbide A, pheophorbide A analog, or pharmaceutically acceptable salt thereof is present in an extract.

7. The method of claim 6, wherein the extract is a plant or algae extract.

8. The method of claim 1, wherein the pheophorbide A, pheophorbide A analog, or pharmaceutically acceptable salt thereof is in purified form.

9. The method of claim 1, wherein the pheophorbide A, pheophorbide A analog, or pharmaceutically acceptable salt thereof is formulated into a pharmaceutical composition.

10. The method of claim 1, wherein the method comprises administering a pharmaceutical composition comprising pheophorbide A.

11. The method of claim 1, wherein the pheophorbide A, pheophorbide A analog, or pharmaceutically acceptable salt thereof is administered intrapulmonary.

12. The method of claim 1, wherein the subject is a human.

13. The method of claim 1, wherein the subject is a non-human animal.

14. The method of claim 13, wherein the non-human animal is a farm animal.

15. The method of claim 13, wherein the non-human animal is a pet.

16-33. (canceled)
